(19) United States
(12) Patent Application Publication
(10) Pub. No.: US 2022/0096519 A1
(43) Pub. Date: Mar. 31, 2022

(54) SOLID PHARMACEUTICAL DOSAGE FORM COMPRISING SPRAY DRIED VITAMIN B12

(71) Applicant: DSM IP ASSETS B.V., Heerlen (NL)

(72) Inventor: Zdravka MISIC, Kaiseraugst (CH)

(21) Appl. No.: 17/619,467

(22) PCT Filed: Jun. 17, 2020
(86) PCT No.: PCT/EP2020/066750

§ 371 Date: Dec. 15, 2021

(30) Foreign Application Priority Data
Jun. 17, 2019 (EP) 19180448.3

(51) Int. Cl. A61K 31/714; A61K 9/20; A61K 31/155

(52) U.S. Cl. CPC A61K 31/714; A61K 9/2013; A61K 31/155; A61K 9/2009

(57) ABSTRACT

The present invention relates to a solid pharmaceutical dosage form comprising at least one spray dried formulation of vitamin B12 and at least one further active pharmaceutical ingredient. Said at least one further active pharmaceutical ingredient is preferably metformin or a pharmaceutical acceptable salt thereof. When manufacturing the dosage form of the invention, uniformity of content is excellent.

### TECHNICAL FIELD

The present invention relates to active pharmaceutical ingredients (API) whose intake cause a depletion of vitamin B12.

### BACKGROUND OF THE INVENTION

During long-term use of certain APIs, the serum vitamin B12 level decreases. An example of such an API is metformin.

A lower than normal absorption of vitamin B12 can be compensated by an increased intake of vitamin B12. Some food (such as liver and kidney) comprises a relatively high amount of vitamin B12. However, for many patients and in particular for vegetarians, an increased consumption of edible offal is not an option.

Crystalline vitamin B12 is commercially available at DSM® Nutritional Products (Switzerland) and can be added to tablets or capsules.

The recommended daily allowance (RDA) of vitamin B12 is about 2 μg. In comparison, the typical maintenance dose of metformin is 2000 mg per day. This corresponds to a weight ratio of 0.002 mg: 2000 mg=1:1.000.000. Making sure that the individual content of vitamin B12 in each capsule or tablet is the same, is challenging.

There is a need for improving the uniformity of content when manufacturing a fixed dose combination (FDC) that comprises a very small amount of vitamin B12 and, in comparison, a very high amount of an API that is known to decrease the serum vitamin B12 level of patients. In particular, there is a need for improving uniformity of content of vitamin B12 when manufacturing a solid oral pharmaceutical dosage form which comprises, in weight, at least ten thousand times more metformin HCl than vitamin B12.

### SUMMARY OF THE INVENTION

The problems underlying the present invention are solved by a solid pharmaceutical dosage form comprising:
a) at least one spray dried formulation of vitamin B12,
b) at least one further active pharmaceutical ingredient such as metformin HCl, and
c) at least one solid pharmaceutically acceptable excipient,
wherein the intake of said at least one further active pharmaceutical ingredient causes depletion of vitamin B12, and/or
wherein the weight ratio between vitamin B12 and said at least one further active pharmaceutical ingredient is preferably from 1:10.000.000 to 1:1.000.

When using, during the manufacturing process, a spray dried formulation of vitamin B12 instead of commercially available vitamin B12 crystals, uniformity of content of the obtained solid pharmaceutical dosage form is dramatically improved. Therefore, the present invention also relates to the use of a spray dried formulation of vitamin B12 for increasing uniformity of content when manufacturing solid pharmaceutical dosage forms that comprise vitamin B12 and at least one further active pharmaceutical ingredient in a weight ratio from 1:10.000.000 to 1:1.000. The invention also relates to the use of a mixture which comprises at least one spray dried formulation of vitamin B12, at least one pharmaceutically acceptable excipient and at least one further active pharmaceutical ingredient for manufacturing an oral solid pharmaceutical dosage form.

The preferred vitamin B12 is cyanocobalamin. If the concentration of cyanocobalamin in the spray dried formulation of vitamin B12 is low, particularly good uniformity of content is achieved. In a preferred embodiment of the invention, the spray dried formulation of vitamin B12 comprises from 0.01 to 1 weight-%, preferably from 0.05 to 0.5 weight-% and most preferably 0.1 weight-% cyanocobalamin, based on the total weight of the spray dried formulation of vitamin B12.

The present invention also relates to a method of manufacturing the herein described solid pharmaceutical dosage form. Said method comprises the step:
spray drying of an aqueous solution that comprises vitamin B12 and at least one excipient

In a preferred embodiment of the invention, the at least one further active pharmaceutical ingredient is metformin or a pharmaceutically acceptable salt thereof such as metformin HCl. The concomitant intake of metformin and vitamin B12 reduces the depletion of vitamin B12 caused by metformin. Therefore, the present invention also relates to the herein described solid pharmaceutical dosage form for use in the treatment or prevention of metformin induced vitamin B12 deficiency.

In a preferred embodiment of the invention, the herein described solid pharmaceutical dosage form is a tablet, a capsule or a powder. For preparing such dosage forms, the addition of at least one solid pharmaceutically acceptable excipient is recommended or even necessary. The at least one solid pharmaceutical excipient is preferably a calcium salt such as calcium phosphate or calcium citrate.

### DETAILED DESCRIPTION OF THE INVENTION

Vitamin B12 is an active pharmaceutical ingredient. The present invention relates to a solid pharmaceutical dosage form that comprises (a) at least one spray dried formulation of vitamin B12, (b) at least one further active pharmaceutical ingredient and (c) at least one solid pharmaceutically acceptable excipient. The preferred vitamin B12 is cyanocobalamin.

### Definitions

The term “comprising” is an open term. Therefore, the herein described solid pharmaceutical dosage form may comprise more than two active pharmaceutical ingredients (APIs). However, the solid pharmaceutical dosage form of the invention comprises preferably two active pharmaceutical ingredients only: vitamin B12 and one further active pharmaceutical ingredient. Vitamin B12 crystals have a vitamin B12 content of at least 98 weight-%, based on the total weight of the crystals, whereas spray dried formulations of vitamin B12 comprise less than 90 weight-% of vitamin B12, based on the total weight of the spray dried formulation. According to the invention, a spray dried formulation of vitamin B12 is used instead of vitamin B12 crystals. Various kinds of spray dried formulations of vitamin B12 are known. The herein described solid pharmaceutical dosage form may comprise more than one kind of spray dried vitamin B12 formulation. However, preferably, the solid pharmaceutical dosage form of the invention comprises one kind of spray dried vitamin B12 formulation only.

The solid pharmaceutical dosage form of the invention comprises at least one solid pharmaceutically acceptable excipient. Preferably, however, the solid pharmaceutical dosage form of the invention comprises at least two, more preferably at least three, and most preferably at least four solid pharmaceutically acceptable excipients.

Thus, an illustrative embodiment of the invention relates to a solid pharmaceutical dosage form comprising:
a) one spray dried formulation of vitamin B12,
b) one further active pharmaceutical ingredient such as metformin HCl, and
c) at least four solid pharmaceutically acceptable excipients,
wherein said spray dried formulation of vitamin B12 comprises less than 90 weight-% of vitamin B12, based on the total weight of the spray dried formulation of vitamin B12, and/or
wherein the weight ratio between vitamin B12 and said at least one further active pharmaceutical ingredient is preferably from 1:10.000.000 to 1:1.000.

In the context of the present invention, the term “solid pharmaceutical dosage form” refers to a dosage form such as a tablet, a capsule and a powder. Powders (such as powders for preparing an oral solution) are typically packaged in a sachet or a stick-pack. Alternatively, powders may be filled into two-piece capsules (e.g. gelatine capsules size 0, 00 or 000). In a preferred embodiment of the invention, the term “solid pharmaceutical dosage form” refers to a solid oral pharmaceutical dosage form selected from the group consisting of tablets, capsules and powders. In an even more preferred embodiment of the invention, the term “solid pharmaceutical dosage form” refers to a compressed tablet.

Vitamin B12 is a well-known water-soluble vitamin. In the context of the present invention, the term “vitamin B12” refers to any vitamer of vitamin B12 and includes vitamin B12 derivatives and/or metabolites of vitamin B12. Preferably, however, the term “vitamin B12” refers to cyanocobalamin. Cyanocobalamin may be produced by fermentation using suitable microorganisms.

“Crystalline vitamin B12” comprises at least 98 weight-% vitamin B12, based on the total weight of the crystals. Preferably, the solid pharmaceutical dosage form of the invention does not comprise any crystalline vitamin B12.

The solid pharmaceutical dosage form of the invention comprises at least one spray dried formulation of vitamin B12. The expression “spray dried formulation of vitamin B12” refers to a powder which is obtainable by spray drying of an aqueous solution that comprises vitamin B12 and at least one excipient, wherein said at least one excipient is preferably selected from the group consisting of sodium citrate, trisodium citrate, citric acid, maltodextrin citric acid and modified food starch. In a preferred embodiment of the invention, the expression “spray dried formulation of vitamin B12” refers to a powder which is obtainable by spray drying an aqueous solution which comprises cyanocobalamin and at least one excipient, wherein said at least one excipient is preferably selected from the group consisting of sodium citrate, trisodium citrate, citric acid, maltodextrin and modified food starch.

Due to the presence of at least one excipient, the spray dried formulation of vitamin B12 comprises less than 90 weight-% of vitamin B12, based on the total weight of the spray dried formulation. The exact concentration of vitamin B12 in the spray dried formulation of vitamin B12 depends on the amount of excipient in the spray dried formulation. Preferably, the spray dried formulation of vitamin B12 of the invention comprises 1 weight-% or less of vitamin B12, based on the total weight of the spray dried formulation, wherein spray dried formulations of vitamin B12 being free of vitamin B12 are excluded. Also preferably, the spray dried formulation of vitamin B12 of the invention is a water-soluble or water-dispersible powder comprising 1 weight-% or less of cyanocobalamin, based on the total weight of the powder, wherein powders being free of vitamin B12 are excluded. In the most preferred embodiment of the invention, the expression “spray dried formulation of vitamin B12” refers to a powder which is obtainable by spray drying an aqueous solution which comprises cyanocobalamin and at least one excipient, wherein said excipient is preferably selected from the group consisting of sodium citrate, trisodium citrate, citric acid, maltodextrin and modified food starch, and wherein said powder comprises 1 weight-% or less of cyanocobalamin, based on the total weight of the powder and wherein powders being free of vitamin B12 are excluded.

The at least one further active pharmaceutical ingredient (API) is preferably an active pharmaceutical ingredient which decreases the serum vitamin B12 level of the patient that is being treated with the API. An example of such a drug is metformin. In most cases, such decrease does not happen instantaneously. Often, the decrease is observed when patient has been taking said API over a period of at least three months or over a period of at least six months or over a period of at least twelve months.

In the context of the present invention, the term “metformin” refers to metformin or to a pharmaceutically acceptable salt thereof. The probably best known pharmaceutically acceptable salt of metformin is metformin HCl. Therefore, in the most preferred embodiment of the invention, the term “metformin” refers to metformin HCl.

Metformin has a poor compactibility and flowability. Therefore, metformin is preferably granulated before tableting. During such granulation process, metformin is transformed into free-flowing, essentially dust-free granules that are easy to compress. In the context of the present invention, the term “granulated metformin” refers to granules comprising metformin or pharmaceutical salt thereof. Preferably, the term “granulated metformin” refers to granules comprising at least 50 weight-% metformin, based on the total weight of the granules, and at least one excipient, wherein said excipient is preferably a binder with or without a lubricant. Suitable binders are listed for example in Arndt et al., “Roll Compaction and Tableting of High Loaded Metformin Formulations Using Efficient Binders”, AAPS PharmSciTech, July 2018, Volume 19, Issue 5, pp 2068-2076. In the context of the present invention, the term “granulated metformin” includes granulated pharmaceutical acceptable salts of metformin such as granulated metformin HCl. Therefore, the term “granulated metformin” may refer to granules comprising at least 50 weight-% metformin HCl, based on the total weight of the granules, and at least one excipient, wherein said excipient is preferably a binder with or without a lubricant. Granulated metformin is commercially available. Metformin granulate DC grade 92.6% as available at Vistin Pharma (Oslo, Norway) comprises magnesium stearate as lubricant. Therefore, in the most preferred embodiment of the invention, the term “granulated metformin” refers to granules comprising magnesium stearate and at least 90 weight-% metformin HCl, based on the total weight of the granules.

In the context of the present invention, the term “solid pharmaceutically acceptable excipient” refers to any pharmaceutically acceptable excipient which is solid at 25° C. and at standard atmosphere (pressure=1.01325 bar). Thus, the term includes acidity regulators, firming agents, release agents, fillers, binders, lubricants, glidants, stabilizers, disintegrants and suspending agents, provided they are solid at 25° C. and at standard atmosphere. Considering that vitamin B12 is pH sensitive, the solid pharmaceutical dosage form of the present invention comprises preferably at least one acidity regulator. According to the invention, the at least one solid pharmaceutically acceptable excipient is preferably a calcium salt or a mixture of calcium salts. Depending on the chosen calcium salt, the calcium salt may be for example a lubricant and/or an acidity regulator.

In the context of the present invention, the term “calcium salt” refers to any pharmaceutically acceptable calcium salt. Thus, the term includes calcium phosphate, calcium carbonate and calcium citrate. The term “calcium citrate” includes monocalcium citrate, dicalcium citrate and tricalcium citrate. Known tricalcium citrate salts include anhydrous calcium citrate (i.e. Ca₃(C₆H₅O₇)₂) and tricalcium dicitrate tetrahydrate (i.e. [Ca₃(C₆H₅O₇)₂(H₂O)₂].2H₂O). The term “calcium phosphate” includes anhydrous calcium phosphate and hydrous calcium phosphate. Known anhydrous calcium phosphates are e.g. anhydrous monocalcium phosphate (Ca(H₂PO₄)₂), anhydrous dicalcium phosphate (CaHPO₄) and anhydrous tricalcium phosphate (Ca₃(PO₄)₂). Calcium carbonate is a chemical compound with the formula CaCO₃.

Microcrystalline cellulose (MCC) is a well-known excipient prepared by acid hydrolysis of cellulose. On industrial scale, MCC is obtained by hydrolysis of wood and/or cotton cellulose using dilute mineral acids. The treated pulp is then rinsed and spray-dried with or without an additional process step such as milling. Numerous types of microcrystalline cellulose (MCC) are available on the market. In the context of the present invention, the term “microcrystalline cellulose” includes any type of microcrystalline cellulose consisting of partially depolymerized cellulose such as the excipients listed in Table 1 of T. Vehovec et al.: “Influence of different types of commercially available microcrystalline cellulose on degradation of perindopril erbumine and enalapril maleate in binary mixtures”, Acta Pharm. 62 (2012), page 518. Also included is silicified microcrystalline cellulose such as PROSOLV® SMCC. In the context of the present invention, the term “silicified microcrystalline cellulose” refers to an excipient comprising microcrystalline cellulose (MCC) and silicon dioxide such as colloidal silicon dioxide (CSD).

“Uniformity of content” ensures that a consistent dose of an active pharmaceutical ingredient is maintained in each individual dosage form (e.g. in each capsule, in each tablet or in each sachet) of a batch. When controlling the quality of e.g. capsules or tablets, uniformity of content is determined. To do so, multiple e.g. capsules or tablets are selected at random and a suitable analytical method is applied to assay the individual content of the active pharmaceutical ingredient in each capsule or tablet. According to European and US pharmacopoeia, from the obtained assay analyses, a relative standard deviation (RSD) and an acceptance value (AV) should be calculated. The lower the RSD, the better the uniformity of content. The calculated acceptance value should be lower than 15. In the context of the present invention, RSD and AV are preferably calculated as set out in “The United States pharmacopeia”, 37th revision, physical tests, <905> Uniformity of Dosage Units, May 1, 2014. The term “uniformity of vitamin B12 content” is used when multiple solid pharmaceutical dosage forms are selected at random and a suitable analytical method is applied to assay the individual content of vitamin B12 in each solid pharmaceutical dosage form.

Solid Pharmaceutical Dosage Form

The present invention relates to a solid pharmaceutical dosage form which comprises vitamin B12. Preferably, said solid pharmaceutical dosage form is a tablet, a capsule or a powder which comprises cyanocobalamin.

The solid pharmaceutical dosage form of the invention comprises preferably from 1 μg to 10 μg cyanocobalamin, more preferably from 2 μg to 9 μg cyanocobalamin and most preferably from 3 μg to 4 μg cyanocobalamin. In case the solid pharmaceutical dosage form is a tablet, the tablet of the present invention comprises preferably from 1 μg to 10 μg cyanocobalamin per tablet, more preferably from 2 μg to 9 μg cyanocobalamin per tablet and most preferably from 3 μg to 4 μg cyanocobalamin per tablet. As for safety, Tolerable Upper Intake Levels (known as ULs) are set for vitamins and minerals when evidence is sufficient. In the case of vitamin B12, there is no UL, as there is no human data for adverse effects from high doses.

The solid pharmaceutical dosage form of the present invention comprises at least one further active pharmaceutical ingredient. Preferably, said at least one further active pharmaceutical ingredient is metformin or a pharmaceutical acceptable salt thereof. The preferred pharmaceutical acceptable salt of metformin is metformin HCl. Typically, the solid pharmaceutical dosage form of the invention has a mass of less than 5 g, preferably of less than 4 g, more preferably of less than 3 g and most preferably of less than 2 g.

Typically, the solid pharmaceutical dosage form of the invention is to be taken twice per day. In a preferred embodiment of the invention, the solid pharmaceutical dosage form comprises vitamin B12 and 1000 mg metformin HCl, 500 mg metformin HCl or 850 mg metformin HCl, wherein the weight ratio between vitamin B12 and metformin HCl is from 1:10.000.000 to 1:1.000, preferably from 1:5.000.000 to 1:2.000 and most preferably from 1:1.000.000 to 1:4.000. In an also preferred embodiment of the invention, the solid pharmaceutical dosage form is a tablet or capsule which comprises 1000 mg metformin HCl or 500 mg metformin HCl and 1 μg to 10 μg cyanocobalamin. In the most preferable embodiment, the solid pharmaceutical dosage form is a tablet which comprises 1000 mg metformin HCl and 1 μg to 4 μg cyanocobalamin. Typically, two of these tablets are given per day to reach a daily dose of 2000 mg metformin HCl.

Ensuring uniformity of vitamin B12 content in each capsule, each tablet or in each portion of powder is extremely difficult. When using commercially available vitamin B12 crystals, uniformity of vitamin B12 content is very poor. In some trials, relative standard deviations (RSD) values of more than 90% were measured.

Uniformity of vitamin B12 content can be tremendously improved when using a spray dried formulation of vitamin B12 instead of using commercially available vitamin B12 crystals. The lower the concentration of vitamin B12 in the spray dried formulation, the higher the uniformity of vitamin B12 content. When using a spray dried formulation of vitamin B12 which contains 1 weight-% cyanocobalamin, based on the total weight of the spray dried formulation of vitamin B12, a relative standard deviation (RSD) of less than 4% can be achieved. When using in the same process a more diluted spray dried formulation of vitamin B12 containing only 0.1 weight-% cyanocobalamin, based on the total weight of the spray dried formulation of vitamin B12, RSD could be further lowered to 2%.

Therefore, the present invention relates preferably to a solid pharmaceutical dosage form comprising:
a) a spray dried formulation of vitamin B12,
b) at least one further active pharmaceutical ingredient, and
c) at least one solid pharmaceutically acceptable excipient,
wherein said spray dried formulation of vitamin B12 comprises preferably from 0.01 to 1 weight-%, more preferably from 0.05 to 0.5 weight-% and most preferably 0.1 weight-% cyanocobalamin, based on the total weight of the spray dried formulation of vitamin B12.

The person skilled in the art knows how to manufacture such spray dried formulations of vitamin B12. In one embodiment, the herein described spray dried formulation of vitamin B12 is produced as disclosed in example 1 of U.S. Pat. No. 5,397,576.

The at least one further active pharmaceutical ingredient is preferably metformin or a pharmaceutically acceptable salt thereof. Therefore, the solid pharmaceutical dosage form of the present invention comprises preferably:
a) a spray dried formulation of vitamin B12,
b) metformin or a pharmaceutically acceptable salt thereof, and
c) at least one solid pharmaceutically acceptable excipient,
wherein said spray dried formulation of vitamin B12 comprises preferably from 0.01 to 1 weight-%, more preferably from 0.05 to 0.5 weight-% and most preferably 0.1 weight-% cyanocobalamin, based on the total weight of the spray dried formulation of vitamin B12.

In an even more preferred embodiment, the solid pharmaceutical dosage form of the present invention comprises:
a) a spray dried formulation of vitamin B12,
b) metformin HCl, and
c) at least one solid pharmaceutically acceptable excipient,
wherein said spray dried formulation of vitamin B12 comprises from 0.01 to 1 weight-%, more preferably from 0.05 to 0.5 weight-% and most preferably 0.1 weight-% cyanocobalamin, based on the total weight of the spray dried formulation of vitamin B12, and/or
wherein the weight ratio between cyanocobalamin and metformin HCl is from 1:10.000.000 to 1:1.000, preferably from 1:5.000.000 to 1:2.000 and most preferably from 1:1.000.000 to 1:4.000.

The at least one solid pharmaceutically acceptable excipient of the invention is preferably an acidity regulator, a firming agent, a release agent, a filler, a binder, a lubricant, a glidant, a stabilizer, a disintegrant and/or a suspending agent. Vitamin B12 is sensitive to pH changes.

Therefore, the solid pharmaceutical dosage form of the present invention comprises preferably:
a) a spray dried formulation of vitamin B12,
b) metformin HCl, and
c) at least one acidity regulator,
wherein said spray dried formulation of vitamin B12 comprises preferably from 0.01 to 1 weight-%, more preferably from 0.05 to 0.5 weight-% and most preferably 0.1 weight-% cyanocobalamin, based on the total weight of the spray dried formulation of vitamin B12, and

wherein said at least one acidity regulator is preferably a calcium salt, and

wherein said calcium salt is preferably selected from the group consisting of calcium phosphate, calcium carbonate and calcium citrate.

In the context of the present invention, calcium phosphate is the preferred acidity regulator. Thus, an also preferred embodiment of the invention relates to a solid pharmaceutical dosage form of the present invention which comprises:
a) a spray dried formulation of vitamin B12,
b) metformin HCl, and
c) at least one calcium salt,
wherein said spray dried formulation of vitamin B12 comprises preferably from 0.01 to 1 weight-%, more preferably from 0.05 to 0.5 weight-% and most preferably 0.1 weight-% cyanocobalamin, based on the total weight of the spray dried formulation of vitamin B12, and
wherein said at least one calcium salt is preferably anhydrous calcium phosphate or hydrous calcium phosphate, and
wherein said anhydrous calcium phosphate is preferably anhydrous monocalcium phosphate (Ca(H₂PO₄)₂), anhydrous dicalcium phosphate (CaHPO₄) or anhydrous tricalcium phosphate (Ca₃(PO₄)₂), and
wherein the weight ratio between vitamin B12 and metformin HCl is from 1:10.000.000 to 1:1.000, preferably from 1:5.000.000 to 1:2.000 and most preferably from 1:1.000.000 to 1:4.000.

In a preferred embodiment of the invention, the solid pharmaceutical dosage form is a tablet. For compressing a tablet, a filler might be needed or is at least recommended. Thus, a preferred embodiment of the invention relates to a tablet comprising:
a) a spray dried formulation of vitamin B12,
b) metformin HCl,
c) at least one calcium salt, and
d) at least one filler
wherein said spray dried formulation of vitamin B12 comprises from 0.01 to 1 weight-%, more preferably from 0.05 to 0.5 weight-% and most preferably 0.1 weight-% cyanocobalamin, based on the total weight of the spray dried formulation of vitamin B12, and
wherein the weight ratio between cyanocobalamin and metformin HCl is from 1:10.000.000 to 1:1.000, preferably from 1:5.000.000 to 1:2.000 and most preferably from 1:1.000.000 to 1:4.000, and
wherein said at least one filler is preferably microcrystalline cellulose or silicified microcrystalline cellulose.

Compressing a tablet is easier when using granulated metformin. Alternatively, the mixture comprising metformin can be granulated before compressing the mixture into tablets. In either case, a binder is used for granulation. Therefore, a preferred embodiment of the invention relates to a tablet comprising:
a) a spray dried formulation of vitamin B12,
b) metformin HCl,
c) at least one calcium salt,
d) at least one filler, and
e) at least one binder,
wherein said spray dried formulation of vitamin B12 comprises from 0.01 to 1 weight-%, more preferably from 0.05 to 0.5 weight-% and most preferably 0.1 weight-% cyanocobalamin, based on the total weight of the spray dried formulation of vitamin B12, and
wherein said at least one filler is preferably microcrystalline cellulose or silicified microcrystalline cellulose.

The most preferred embodiment of the invention relates to a tablet comprising:
a) a spray dried formulation of vitamin B12,
b) metformin HCl, and
c) at least one calcium salt,
d) at least one filler, and
e) at least one binder,
wherein said spray dried formulation of vitamin B12 comprises less than 1 weight-% cyanocobalamin, based on the total weight of the spray dried formulation of vitamin B12, and
wherein the weight ratio between cyanocobalamin and metformin HCl is from 1:10.000.000 to 1:1.000, preferably from 1:5.000.000 to 1:2.000 and most preferably from 1:1.000.000 to 1:4.000, and
wherein said at least one filler is preferably microcrystalline cellulose or silicified microcrystalline cellulose.

An alternative embodiment of the invention relates to a tablet comprising:
a) a spray dried formulation of vitamin B12,
b) granulated metformin HCl,
c) at least one calcium salt, and
d) at least one filler
wherein said spray dried formulation of vitamin B12 comprises from 0.01 to 1 weight-%, more preferably from 0.05 to 0.5 weight-% and most preferably 0.1 weight-% cyanocobalamin, based on the total weight of the spray dried formulation of vitamin B12, and
wherein the weight ratio between cyanocobalamin and metformin HCl is from 1:10.000.000 to 1:1.000, preferably from 1:5.000.000 to 1:2.000 and most preferably from 1:1.000.000 to 1:4.000, and
wherein said at least one filler is preferably microcrystalline cellulose or silicified microcrystalline cellulose.

Method of Manufacturing the Solid Pharmaceutical Dosage Form of the Present Invention

In one embodiment of the invention, the pharmaceutical dosage form is a powder. Such powder can be prepared by mixing the components of the powder. Optionally, the powder can then be filled into capsule shells or into sachets or can be compressed into tablets. Thereby, content uniformity is improved if a spray dried formulation of vitamin B12 is used.

Suitable spray dried formulation of vitamin B12 are commercially available as “Vitamin B12 1% SD” or “Vitamin B12 0.1% WS” from DSM® Nutritional Products (Switzerland). “Vitamin B12 1% SD” comprises 1 weight-% cyanocobalamin, based on the total weight of the respective product, whereas “Vitamin B12 0.1% WS” comprises 0.1 weight-% cyanocobalamin, based on the total weight of the respective product. According to the present invention, “Vitamin B12 0.1% WS” is the preferred spray dried formulation of vitamin B12.

The present invention relates to the use of a spray dried formulation of vitamin B12 for increasing uniformity of content when manufacturing solid pharmaceutical dosage forms which comprise vitamin B12 and at least one further active pharmaceutical ingredient in a weight ratio from 1:10.000.000 to 1:1.000, preferably from 1:5.000.000 to 1:2.000 and most preferably from 1:1.000.000 to 1:4.000. In a preferred embodiment of the invention, a spray dried formulation of vitamin B12 which comprises from 0.01 to 1 weight-%, preferably from 0.05 to 0.5 weight-% and most preferably 0.1 weight-% cyanocobalamin, based on the total weight of the spray dried formulation of vitamin B12, is used for increasing uniformity of content when manufacturing solid pharmaceutical dosage forms which comprise vitamin B12 and metformin or a pharmaceutically acceptable salt thereof. Most preferably, the present invention relates to the use of a spray dried formulation which comprises 0.1 weight-% cyanocobalamin based on the total weight of the spray dried formulation of vitamin B12 for manufacturing a solid oral pharmaceutical dosage form which comprises 500 mg, 850 mg or 1000 mg metformin HCl.

The present invention also relates to a method of manufacturing a solid pharmaceutical dosage form as herein described, said method comprising the step:
spray drying of an aqueous solution that comprises vitamin B12 and at least one auxiliary compound, wherein said at least one auxiliary compound is preferably selected from the group consisting of sodium citrate, trisodium citrate, citric acid, maltodextrin citric acid and modified food starch.

The preferred method of manufacturing a solid pharmaceutical dosage form as herein described comprises the step:
providing a powder by mixing at least one spray dried formulation of vitamin B12, at least one further active pharmaceutical ingredient and at least one solid pharmaceutically acceptable excipient, wherein the weight ratio between vitamin B12 and said at least one further active pharmaceutical ingredient is from 1:10.000.000 to 1:1.000, preferably from 1:5.000.000 to 1:2.000 and most preferably from 1:1.000.000 to 1:4.000.

An even more preferred method of manufacturing a solid pharmaceutical dosage form as herein described comprises the steps:
providing a mixture comprising at least one spray dried formulation of vitamin B12, at least one further active pharmaceutical ingredient and at least one calcium salt, at least one filler and at least one binder, wherein the weight ratio between vitamin B12 and said at least one further active pharmaceutical ingredient is from 1:10.000.000 to 1:1.000, preferably from 1:5.000.000 to 1:2.000 and most preferably from 1:1.000.000 to 1:4.000
granulating the provided mixture
optionally compressing the obtained granulate into at least one tablet.

An also preferred method of manufacturing a solid pharmaceutical dosage form as herein described comprises the steps:
providing a mixture comprising at least one spray dried formulation of vitamin B12, metformin HCl and at least one calcium salt, at least one filler and at least one binder, wherein the weight ratio between vitamin B12 and metformin HCl is from 1:10.000.000 to 1:1.000, preferably from 1:5.000.000 to 1:2.000 and most preferably from 1:1.000.000 to 1:4.000
granulating the provided mixture
optionally compressing the obtained granulate into at least one tablet.

An alternative method of manufacturing a solid pharmaceutical dosage form as herein described comprises the steps:
providing a mixture comprising at least one spray dried formulation of vitamin B12, granulated metformin HCl and at least one calcium salt, at least one filler and at least one binder, wherein the weight ratio between vitamin B12 and metformin HCl is from 1:10.000.000 to 1:1.000, preferably from 1:5.000.000 to 1:2.000 and most preferably from 1:1.000.000 to 1:4.000
optionally compressing the obtained mixture into at least one tablet.

The most preferred method of manufacturing a solid pharmaceutical dosage form as herein described comprises the steps:
providing a mixture comprising at least one spray dried formulation of vitamin B12, at least one further active pharmaceutical ingredient and at least one calcium salt, microcrystalline cellulose and at least one binder, wherein the weight ratio between vitamin B12 and said at least one further active pharmaceutical ingredient is from 1:10.000.000 to 1:1.000, preferably from 1:5.000.000 to 1:2.000 and most preferably from 1:1.000.000 to 1:4.000
granulating the provided mixture
optionally compressing the obtained granulate into at least one tablet.

Medical Use and Method of Treatment

The present invention also relates to the herein described solid pharmaceutical dosage form for use as a medicament. In case the solid pharmaceutical dosage form comprises metformin, the present invention also relates to the herein described a solid pharmaceutical dosage form for use in the treatment of a patient who is in need of metformin. Thus, one embodiment of the invention relates to a solid pharmaceutical dosage form comprising:
a) at least one spray dried formulation of vitamin B12,
b) metformin or a pharmaceutically acceptable salt thereof, and
c) at least one solid pharmaceutically acceptable excipient, for use in the treatment of a patient who is in need of metformin.

A patient suffering from diabetes may be in need of metformin. Therefore, the present invention also relates to the herein described a solid pharmaceutical dosage form comprising metformin for use in the treatment of diabetes. Thus, one embodiment of the invention relates to a solid pharmaceutical dosage form comprising:
a) at least one spray dried formulation of vitamin B12,
b) metformin or a pharmaceutically acceptable salt thereof, and
c) at least one solid pharmaceutically acceptable excipient, for use in the treatment of diabetes, preferably for use in the treatment of a vegan or vegetarian patient suffering from diabetes. Vegan and vegetarian patients are particularly prone to lower than normal serum vitamin B12 levels.

The present invention also relates to a method for the treatment of diabetes, said method comprising the step of administering the herein described solid pharmaceutical dosage form. A preferred embodiment of the invention relates to a method for the treatment of diabetes, said method comprising the step of administering a solid pharmaceutical dosage form, wherein said solid pharmaceutical dosage form comprises:
a) at least one spray dried formulation of vitamin B12,
b) metformin or a pharmaceutical salt thereof, and
c) at least one solid pharmaceutically acceptable excipient.

When applying this treatment, metformin induced vitamin B12 deficiency is prevented, alleviated or treated.

### EXAMPLES

### Example 1

In example 1, three different kinds of tablets were prepared. Each tablet comprised 549.9 mg calcium phosphate (anhydrous, available at Emcompress®) and 0.0078 mg vitamin B12. Apart from the source of vitamin B12, the different kinds of tablets were identical.

To investigate the impact on content uniformity, the following three different kinds of vitamin B12 were tested:
Vitamin B12 cryst. (crystalline vitamin B12, available at DSM® Nutritional Products)
Vitamin B12 1% SD (spray dried formulation of vitamin B12, available at DSM® Nutritional Products)
Vitamin B12 0.1% WS (spray dried formulation of vitamin B12, available at DSM® Nutritional Products)

Tablets were compressed with a Korsch XL 100 rotary tableting machine (Korsch AG, Berlin, Germany) using an oblong punch of 22×9 mm and compression force of 20 kN.

Vitamin B12 content uniformity was then evaluated via the standard deviation RSD (%) and acceptance value (AV) calculated from 10 individual assay determinations (HPLC analysis conducted by Eurofins®, Germany).

As shown at below TABLE 1, the relative standard deviations (RSD) values relating to the two spray dried formulations of vitamin B12 were below 5%, indicating acceptable content uniformity and hence homogeneous distribution of Vitamin B12 in the tablets. In contrast, content uniformity relating to vitamin B12 crystalline was extremely poor.

TABLE 1

| | | tablet 1 | tablet 2 | tablet 3 |
|---|---|---|---|---|
| weight of tablet (mg) | | 1560.8 | 1553.9 | 1538.3 |
| calcium phosphate | | 549.9 | 549.9 | 549.9 |
| anhydrous (mg/tablet) | | | | |
| source of vitamin B12 | | vitamin B12 | vitamin B12 | vitamin B12 |
| | | cryst. | 1% SD | 0.1% WS |
| amount of source of | | 0.0078 | 0.78 | 7.8 |
| vitamin B12 (mg/tablet) | | | | |
| amount of vitamin | | 0.0078 | 0.0078 | 0.0078 |
| B12 per tablet (mg/tablet) | | | | |
| vitamin | RSD (%) | 90.8 | 3.3 | 2.0 |
| B12 | Acceptance | 270.8 | 8.2 | 11.6 |
| content | value (AV) | fail | pass | pass |
| uniformity | rating | extremely | good | good |
| | | poor | | |

RSD and AV were calculated as set out in “The United States pharmacopeia, 37th revision, May 1, 2014, physical tests, <905> Uniformity of Dosage Units”.

### Example 2

In Example 2, tablets comprising (a) spray dried formulation of vitamin B12, (b) metformin and (c) several solid pharmaceutically acceptable excipients were prepared. Granulated Metformin DC 92.6% available at Vistin Pharma (Oslo, Norway) was used. As solid pharmaceutically acceptable excipients, two different kinds of calcium salts were used. The obtained tablets are very hard, disintegration time was short, and no capping was observed during tabletting process.

Below TABLE 2 shows the composition of tablets of the invention.

TABLE 2

| | |
|---|---|
| Tablet 1 | Tablet 2 |

| | | | | |
|---|---|---|---|---|
| Metformin DC 92.6% | 863.9 | mg | 863.9 | mg |
| Vitamin B12 (spray-dried) | 1.56 | mg | 1.56 | mg |

| | | | |
|---|---|---|---|
| tricalcium dicitrate tetrahydrate | 434.78 | mg | — |

| | | | |
|---|---|---|---|
| anhydrous calcium citrate | — | 423.73 | mg |

| | | | | |
|---|---|---|---|---|
| Aerosil 200 | 4.28 | mg | 4.28 | mg |
| Magnesium stearate | 2.5 | mg | 2.5 | mg |
| Prosolv SMCC90 | 192.95 | mg | 204 | mg |
| Total weight of tablet | 1500 | mg | 1500 | mg |

Both tablets comprised 863.9 mg*92.6%=800 mg metformin. Both tablets comprised 1.56 mg*1%=0.0156 mg cyanocobalamin. Thus, the weight ratio cyanocobalamin:metformin was in tablets 1:51.282.

Both tablets are suitable for treating patients which are suffering from a medical indication that can be treated with metformin. Thus, both tablets are for use in the treatment of diabetes.

1. Solid pharmaceutical dosage form comprising:
a) at least one spray dried formulation of vitamin B12,
b) at least one further active pharmaceutical ingredient, and
c) at least one solid pharmaceutically acceptable excipient,
wherein the weight ratio between vitamin B12 and said at least one further active pharmaceutical ingredient is from 1:10.000.000 to 1:1.000.

2. Solid pharmaceutical dosage form according to claim 1, wherein said at least one solid pharmaceutical excipient is an acidity regulator, a firming agent, a release agent, a filler, a binder, a lubricant, a glidant, a stabilizer, a disintegrant or a suspending agent, and wherein said at least one pharmaceutical excipient is preferably an acidity regulator.

3. Solid pharmaceutical dosage form according to claim 1, wherein said at least one solid active pharmaceutical excipient is a calcium salt, and wherein said calcium salt is preferably calcium phosphate, and
wherein said calcium phosphate is preferably anhydrous calcium phosphate or hydrous calcium phosphate, and
wherein said anhydrous calcium phosphate is preferably anhydrous monocalcium phosphate (Ca(H₂PO₄)₂), anhydrous dicalcium phosphate (CaHPO₄) or anhydrous tricalcium phosphate (Ca₃(PO₄)₂).

4. Solid pharmaceutical dosage form according to claim 1, wherein said at least one further active pharmaceutical ingredient is metformin or a pharmaceutically acceptable salt thereof, and wherein said at least one further active pharmaceutical ingredient is preferably metformin HCl.

5. Solid pharmaceutical dosage form according to claim 1, wherein the spray dried formulation of vitamin B12 is obtainable by spray drying of an aqueous solution that comprises vitamin B12 and at least one auxiliary compound, and
wherein said at least one auxiliary compound is preferably selected from the group consisting of sodium citrate, trisodium citrate, citric acid, maltodextrin citric acid and modified food starch.

6. Solid pharmaceutical dosage form according to claim 1, wherein said spray dried formulation of vitamin B12 comprises from 0.01 to 1 weight-%, preferably from 0.05 to 0.5 weight-% and most preferably 0.1 weight-% cyanocobalamin, based on the total weight of the spray dried formulation of vitamin B12.

7. Solid pharmaceutical dosage form according to claim 1, wherein said spray dried formulation of vitamin B12 is water-dispersible or water-soluble, and wherein said spray dried formulation of vitamin B12 is preferably water-soluble.

8. Solid pharmaceutical dosage form according to claim 1, wherein said solid pharmaceutical dosage form is an oral solid pharmaceutical dosage form, and
wherein said oral solid pharmaceutical dosage form is preferably a tablet or a capsule, and wherein said tablet is preferably a compressed tablet.

9. Method of manufacturing a solid pharmaceutical dosage form which comprises vitamin B12 and at least one further active pharmaceutical ingredient, said method comprising the step:
spray drying of an aqueous solution that comprises vitamin B12 and at least one excipient.

10. Method according to claim 9, wherein the solid pharmaceutical dosage form is prepared.

11. Use of a spray dried formulation of vitamin B12 for increasing uniformity of content when manufacturing solid pharmaceutical dosage forms which comprise vitamin B12 and at least one further active pharmaceutical ingredient in a weight ratio from 1:10.000.000 to 1:1.000.

12. Use according to claim 11, wherein the solid pharmaceutical dosage form is the solid pharmaceutical dosage form according to any one of claims 1 to 8.

13. Solid pharmaceutical dosage form comprising:
a) at least one spray dried formulation of vitamin B12,
b) metformin or a pharmaceutically acceptable salt thereof, and
c) at least one solid pharmaceutically acceptable excipient,
for use in the treatment of a patient who is in need of metformin.

14. Solid pharmaceutical dosage form comprising:
a) at least one spray dried formulation of vitamin B12,
b) metformin or a pharmaceutically acceptable salt thereof, and
c) at least one solid pharmaceutically acceptable excipient,
for use in the treatment of diabetes.

15. Solid pharmaceutical dosage form for use according to claim 13, wherein said solid pharmaceutical dosage form is the solid pharmaceutical dosage.
